(19) United States
(12) Patent Application Publication
(10) Pub. No.: US 2003/0135194 A1
(43) Pub. Date: Jul. 17, 2003

(54) Cartridge with an excentric puncturing area

(76) Inventors: Henrik Sonderskov Klint, Lyngby (DK); Jens Moller Jensen, Copenhagen (DK); Lars Morch Groth, Fredensborg (DK); Andre Larsen, Dragor (DK); Lars Skouboe, Aalborg (DK)

Correspondence Address: Reza Green, Esq., Novo Nordisk Pharmaceuticals, Inc., 100 College Road West, Princeton, NJ, 08540, US

(21) Appl. No.: 10/265,830

(22) Filed: Oct. 7, 2002

Related U.S. Application Data
(60) Provisional application No. 60/329,778, filed on Oct. 16, 2001.

(30) Foreign Application Priority Data
Oct. 8, 2001 (DK) PA 2001 01479

(51) Int. Cl. A61B 19/00; B65D 47/00

(52) U.S. Cl. 604/415; 215/247; 215/DIG. 3

(57) ABSTRACT

A cartridge for holding a liquid and for use in a medical delivery device has an elongate barrel provided with a central axis and having a first opening at a first end thereof and a second opening at a second end thereof. The first opening is sealed off by a first sealing means provided with a puncturing area to be punctured by a conduit, such as a hollow needle, and the second opening is sealed off by a stopper that is in intimate contact with a general inner surface of the barrel and is movable within the barrel towards the first sealing means. The puncturing area of the first sealing means is located radially outside the general inner surface of the barrel.

### CROSS REFERENCE OF RELATED APPLICATIONS

This Application claims the benefit of priority under 35 U.S.C. §119 of U.S. Provisional Application No. 60/329,778, filed on Oct. 16, 2001 and Danish Application PA 2001 01479, filed on Oct. 8, 2001; the entire contents of both are hereby incorporated by reference.

### BACKGROUND OF THE INVENTION

The invention relates to a cartridge for a liquid and for use in a medical delivery device. Such cartridges are well-known for holding various pharmaceutical liquids such as anaesthetics, insulin, etc, and are pre-filled by the manufacturer of the pharmaceutical liquid. Such pre-filled cartridges have a number of advantages over the prior art vial and syringe system in which the pharmaceutical liquid is drawn from a vial into a syringe by a penetrable membrane provided on the vial being punctured by a hollow needle provided on the syringe. The main advantages are that the risk of contamination of the pharmaceutical liquid is minimized, that the dosing of the injection is more easily set and that the waste of injectable liquid is reduced.

The pre-filled cartridges are usable in connection with a variety of medical delivery devices, such as injection devices and infusion devices. In the following the invention will be discussed in relation an injection device comprising a hollow needle that punctures the puncturing area of the cartridge. It is clear, however, that the cartridge is equally suitable in connection with other injection devices and with infusion devices in which the conduit that punctures the puncturing area is not a hollow needle but e.g. a hollow plastics tube or the like. The cartridge according to the invention is therefore not restricted for use in connection with injection devices, but for general use in connection with medical delivery devices.

The known pre-filled cartridges are provided as a cylindrical barrel having a penetrable membrane at a first end thereof and a movable plunger or stopper provided within the barrel in intimate contact with the inner surface of the barrel and being movable towards the first end of the barrel. The barrel is normally made of glass and the penetrable membrane is made of rubber and is secured to the first end of the barrel by means of metal cap that is crimped around the membrane and a collar provided on the glass barrel. The metal cap has a central aperture exposing a puncturing area at the central portion of the membrane for being penetrated or punctured by a conduit, such as a hollow needle. The puncturing area has a certain size in order to ensure that the hollow needle punctures the membrane even if the needle is located slightly excentrically in relation to the barrel.

In use the cartridge is positioned in an injection device comprising a cartridge holder provided with a plunger rod at one end that can be engaged with the stopper within the cartridge, and a needle fitting at the other end adapted to receive a double needle assembly (i.e. a hypodermic needle which is fixed to a support with a first end of the hollow needle extending away from the support in a first direction and a second end of the hollow needle extending away form the support in the opposite direction). After the cartridge has been positioned in the injection device, a double needle assembly is attached to the fitting whereby the first end of the hollow needle punctures the membrane of the cartridge. The liquid can now be expelled from the cartridge through the hollow needle by moving the plunger rod, which is engaged with the movable stopper, towards the membrane.

After the hollow needle has been mounted on the injection device, it extends a certain distance into the barrel. In principle, the needle need only just penetrate the membrane; however, since the membrane is made of rubber it flexes resiliently when the needle is forced against the outer surface of the membrane before puncturing the membrane. Even when the needle has punctured the membrane and is forced further forwards to its final position, the membrane flexes resiliently inwards. This behaviour of the membrane means that the needle must have a certain length in order to ensure that the membrane is fully penetrated. It also means, however, that the needle extends a certain distance into the barrel, e.g. up to 3-4 mm. Since it is undesirable that the stopper touches the needle, the cartridge or the injection device must be provided with means that ensure that the stopper stops its movement towards the membrane a few millimetres above the needle end.

This means, however, that a certain amount of liquid in the cartridge will never be able to be expelled since it is positioned in the space around the needle and between the needle end and the stopper in its end position. This liquid is therefore discarded together with the cartridge after use. The amount of liquid left in the cartridge may be small, but if the number of cartridges is very large the total amount of discarded liquid may amount to several litres or tons a year. It would be very desirable for the manufacturer of the pharmaceutical liquid if this amount of wasted liquid could be reduced.

### SUMMARY OF THE INVENTION

The present invention provides for a cartridge comprising an elongate barrel provided with a central axis and having a first opening at a first end thereof and a second opening at a second end thereof, said first opening being sealed off by a first sealing means provided with a puncturing area to be punctured by a conduit, such as a hollow needle, and said second opening being sealed off by a stopper that is in intimate contact with a general inner surface of the barrel and is movable within the barrel towards said first sealing means.

In some embodiments of the present invention the amount of non-expellable liquid is reduced compared with the known cartridges. This is achieved by arranging the cartridge such that the puncturing area of the first sealing means is located radially outside the general inner surface of the barrel.

Thereby, the stopper, which is in intimate contact with the inner surface of the barrel, is never able to engage the hollow needle since it simply passes by the needle when it is displaced within the barrel. This means that the stopper can reach the end of the barrel when it is fully depressed, whereby almost all liquid is expelled except for a small amount due to the necessary small space that must be provided above the membrane in order to give space for the end of the hollow needle. This space can, however, be very small since the end position of the stopper need not be taken into account.

In a first embodiment the barrel is provided with an expanded part at the first end and said first opening is provided in the expanded part. The expanded part is relatively small in order to minimize the volume with non-expellable liquid.

In this embodiment the first sealing means may be arranged to extend in a plane perpendicular to a central axis of the barrel or in a plane parallel with a central axis of the barrel. The two different extensions of the first sealing means calls for two possibilities of needle attachment, i.e. parallel with the central axis of the barrel or perpendicular to the central axis of the barrel.

In another embodiment according to the invention the first opening is provided in a sidewall of the barrel, and the first sealing means extends annularly around the barrel forming an annular cavity connected to the first opening. In this embodiment the puncturing area is located annularly around the barrel which means that no specific orientation of the barrel is required in order to ensure proper needle attachment.

In this embodiment there is preferably provided an annular collar on the barrel immediately adjacent the first opening, and the first sealing means is arranged to extend from the annular collar across the first opening to engage an outer circumference of the barrel.

Preferably the barrel is made of plastics and the first sealing means is secured to the barrel by means of gluing or welding. Furthermore, the barrel may generally have a cylindrical shape.

### BRIEF DESCRIPTION OF THE DRAWINGS

The invention will be discussed in further detail below with reference to the drawing in which

FIGS. 1a and 1b show a cartridge according to the prior art,

FIGS. 2a and 2b show a cartridge according to the invention in a first embodiment,

FIGS. 3a and 3b show a cartridge according to the invention in a second embodiment, and

FIGS. 4a and 4b show a cartridge according to the invention in a third embodiment.

### DETAILED DESCRIPTION OF THE INVENTION

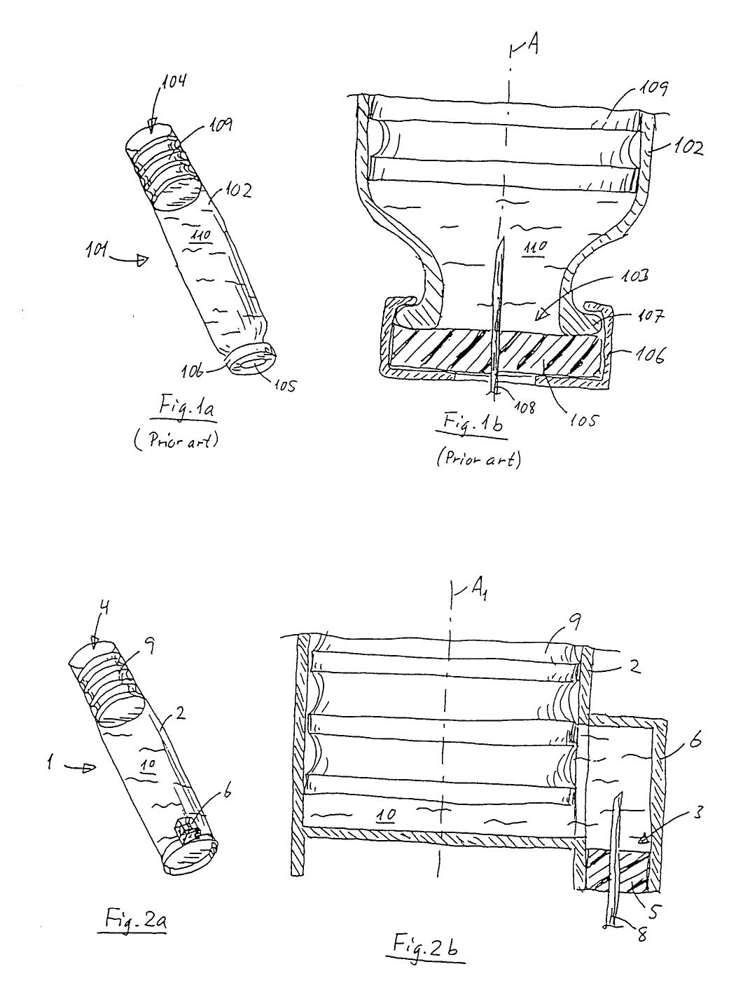

FIG. 1a shows a cartridge 101 according to the prior art and FIG. 1b shows an enlargement of one end of the cartridge 101 shown in FIG. 1a. The cartridge 101 consists of a cylindrical glass barrel 102 with a first opening 103 at the first end thereof and a second opening 104 at the second end thereof. The first opening 103 is sealed off by a first sealing means in form of a puncturable rubber membrane 105 that is secured to the glass barrel 102 by means of a metal cap 106 that is crimped around the membrane 105 and a collar 107 provided on the glass barrel 102. The metal cap 106 has a central aperture exposing the central portion of the membrane 105 as a puncturing area to be punctured by a hollow needle 108. The puncturing area of the membrane 105 is positioned centrally in relation to a central axis A of the cartridge.

The second end of the glass barrel 102 is sealed off by a movable plunger or stopper 109 which is positioned within the glass barrel 102 and is in intimate contact with the inner surface of the glass barrel 102.

FIG. 1a shows the cartridge 101 in its initial state filled with an injectable liquid 110 and with the stopper 109 in its rearmost position. FIG. 1b shows a cross-section of the first end of the cartridge 101 with the stopper 109 in its end position where no more liquid 110 can be expelled from the cartridge 101. It appears from FIG. 1b that there remains a considerable amount of liquid 110 in the cartridge 101 which must be discarded together with the cartridge 101 itself.

FIG. 2a shows a cartridge 1 according to the invention in a first embodiment and FIG. 2b shows an enlargement of one end of the cartridge 1 shown in FIG. 2a. The cartridge 1 comprises a generally cylindrical barrel 2, which is preferably made of plastics, with a central axis A1 and having a first opening 3 at the first end thereof and a second opening 4 at the second end thereof. The first opening 3 is provided in an expanded part 6 of the barrel 2 and is sealed off by a first sealing means in form of a puncturable rubber membrane 5 that is secured to the barrel 2, e.g. by gluing or welding. Other means for securing the membrane 5 to the barrel 2 may be used if applicable, e.g. by crimping a metal cap around the membrane and a collar provided on the barrel. The membrane 5 is exposed and has a puncturing area that is located radially outside the general inner surface of the barrel 2. The membrane 5 can be punctured by a hollow needle 8 as shown in FIG. 2b.

It should be mentioned that the term “the general inner surface of the barrel”, when used in this specification, is meant to mean the inner surface of the barrel with which the stopper is in intimate contact, including any virtual surface where a part the surface has been removed, e.g. for providing liquid passage to a space located outside the barrel.

The second end of the barrel 2 is sealed off by a movable plunger or stopper 9 which is positioned within the barrel 2 and is in intimate contact with the general inner surface thereof.

Again, FIG. 2a shows the cartridge 1 in its initial state filled with an injectable liquid 10 and with the stopper 9 in its rearmost position. FIG. 2b shows a cross-section of the first end of the cartridge 1 with the stopper 9 near its end position having expelled almost all liquid 10 from the cartridge 1. Due to the structure of the barrel 1 and in particular to its expanded part 6, which places the puncturing area radially outside the general inner surface of the barrel 2, it is possible to expel almost all liquid 10 from the cartridge 1. In fact, only an amount of liquid 10 corresponding to the volume of the expanded part 6 will not be expellable. The amount of liquid 10 to be discarded together with the cartridge 1 itself is thereby reduced considerably compared with the prior art cartridges.

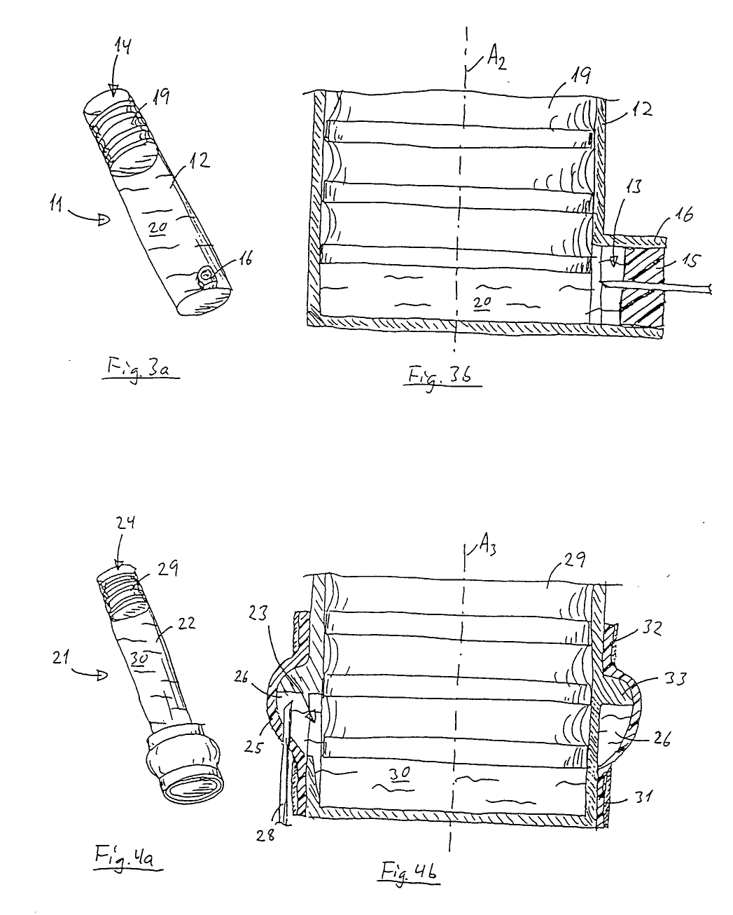

FIG. 3a shows a cartridge 11 according to the invention in a second embodiment and FIG. 3b shows an enlargement of one end of the cartridge 11 shown in FIG. 3a. Again, the cartridge 11 comprises a barrel 12, which is preferably made of plastics, with a central axis A₂ and having a first opening 13 at the first end thereof and a second opening 14 at the second end thereof. The first opening 13, which in this embodiment is provided radially in relation to the barrel 12, is provided in an expanded part 16 of the barrel 12 and is sealed off by a first sealing means in form of a puncturable rubber membrane 15 that is secured to the barrel 12, e.g. by gluing or welding. Other means for securing the membrane 15 to the barrel 12 may be used if applicable, e.g. by crimping a metal cap around the membrane and a collar provided on the barrel. The membrane 15 is exposed and has a puncturing area that is located radially outside the general inner surface of the barrel 2. The membrane 15 can be punctured by a hollow needle 18 as shown in FIG. 2b.

The second end of the barrel 12 is sealed off by a movable plunger or stopper 19 which is positioned within the barrel 12 and is in intimate contact with the general inner surface thereof.

Again, FIG. 3a shows the cartridge 11 in its initial state filled with an injectable liquid 20 and with the stopper 19 is in its rearmost position. FIG. 3b shows a cross-section of the first end of the cartridge 11 with the stopper 19 near its end position having expelled almost all liquid 20 from the cartridge 11. Due to the structure of the barrel 11 and in particular to its expanded part 16, which places the puncturing area radially outside the general inner surface of the barrel 12, it is possible to expel almost all liquid 20 from the cartridge 11. In fact, only an amount of liquid 20 corresponding to the volume of the expanded part 16 will not be expellable. The amount of liquid 20 to be discarded together with the cartridge 11 itself is thereby reduced considerably in comparison with the prior art cartridges.

The main difference between the cartridge 1 shown in FIGS. 1a and 1b and the cartridge 11 shown in FIGS. 2a and 2b is the exposure of the membranes 5 and 15 and thereby the location of the puncturing area. In the cartridge 1 the orientation of the membrane 5 calls for an axial puncturing by the hollow needle 8, whereas the orientation of the membrane 15 in the cartridge 11 calls for a radial puncturing by the hollow needle 18. Each of these two cartridges 1,11 complies with the object of the invention in the same manner, but they are suitable for use with different injection devices having different orientations of the hollow needle.

FIG. 4a shows a cartridge 21 according to the invention in a third embodiment and FIG. 4b shows an enlargement of one end of the cartridge 21 shown in FIG. 3a. Again, the cartridge 21 comprises a barrel 22, which is preferably made of plastics, with a central axis A₃ and a first opening 23 at the first end thereof and a second opening 24 at the second end thereof. The first opening 23, which in this embodiment is provided radially in the barrel wall, opens into a ring-shaped cavity 26 that outwardly is sealed off by a first sealing means in form of an annular puncturable rubber membrane 25 that is secured to the barrel 22 by means of two metal rings 31,32. Other means for securing the membrane 25 to the barrel 22 may be used if applicable, e.g. gluing or welding. The barrel 22 is provided with an outwardly extending annular collar 33 that forces the membrane 25 away from the outer surface of the barrel 22 in order to provide the ring-shaped cavity 26. The membrane 25 is exposed annularly, i.e. the puncturing area, which is located radially outside the general inner surface of the barrel 22, may be punctured by a hollow needle 28 as shown in FIG. 4b anywhere along the circumference of the barrel 22.

The second end of the barrel 22 is sealed off by a movable plunger or stopper 29 which is positioned within the barrel 22 and is in intimate contact with the general inner surface thereof.

Again, FIG. 4a shows the cartridge 21 in its initial state filled with an injectable liquid 30 and with the stopper 29 is in its rearmost position. FIG. 4b shows a cross-section of the first end of the cartridge 21 with the stopper 29 near its end position having expelled almost all liquid 30 from the cartridge 21. Due to the structure of the barrel 21 and in particular to the shaping of the annular cavity 26 it is possible to expel almost all liquid 30 from the cartridge 21. In fact, only an amount of liquid 30 corresponding to the volume of the annular cavity 26 will not be expellable. The amount of liquid 30 which must be discarded together with the cartridge 21 itself is thereby reduced considerably in comparison with the prior art cartridges.

In relation to the embodiments shown in FIGS. 2a, 2b, 3a and 3b, in which the cartridge 1,11 must be specific angularly oriented in relation to the injection device in order to ensure that the hollow needle 8,18 punctures the membrane 5,15, the embodiment shown in FIGS. 4a and 4b does not require any specific angular orientation of the cartridge 21 since the puncturing area of the membrane 25 extends annularly around the barrel 22.

In FIG. 4b the hollow needle 28 is shown with an axial extension; it is clear, however, that in this embodiment the hollow needle 28 might have a radial or an angled extension in relation to the barrel 22.

The invention has been described with reference to different embodiments. Each embodiment requires a specific structure of the medical delivery device in which the specific cartridge is employed in order to ensure proper fitting between the medical delivery device and the cartridge. Therefore, each specific embodiment codes for a specific medical delivery device, and the risk of employing a wrong cartridge is eliminated.

We claim:

1. A cartridge for a liquid and for use in a medical delivery device, said cartridge comprising an elongate barrel provided with a central axis and having a first opening at a first end thereof and a second opening at a second end thereof, said first opening being sealed off by a first sealing means provided with a puncturing area to be punctured by a conduit, such as a hollow needle, and said second opening being sealed off by a stopper that is in intimate contact with a general inner surface of the barrel and is movable within the barrel towards said first sealing means, wherein said puncturing area of said first sealing means is located radially outside the general inner surface of the barrel.

2. A cartridge according to claim 1, wherein the barrel is provided with an expanded part at the first end and that said first opening is provided in the expanded part.

3. A cartridge according to claim 2, wherein said first sealing means extends in a plane perpendicular to the central axis of the barrel.

4. A cartridge according to claim 2, wherein said first sealing means extends in a plane parallel with the central axis of the barrel.

5. A cartridge according to claim 1, wherein the first opening is provided in a sidewall of the barrel, and that the first sealing means extends annularly around the barrel forming an annular cavity connected to the first opening.

6. A cartridge according to claim 5, wherein an annular collar is provided on the barrel immediately adjacent the first opening, and that the first sealing means extends from the annular collar across the first opening to engage an outer circumference of the barrel.

7. A cartridge according to any one of claim 1, wherein the barrel is made of plastics.

8. A cartridge according to claim 7, wherein the first sealing means is secured to the barrel by means of gluing or welding.

9. A cartridge according to any one of claim 1, wherein the barrel has a generally cylindrical shape.
